(19) United States
(12) Patent Application Publication
(10) Pub. No.: US 2023/0104795 A1
(43) Pub. Date: Apr. 6, 2023

(54) METHODS FOR MANAGING ONE OR MORE UNCORRELATED ELEMENTS IN DATA AND DEVICES THEREOF

(71) Applicant: Mitchell International, Inc., San Diego, CA (US)

(72) Inventors: Mary Cassol, San Diego, CA (US); Vicki Dunbar, San Diego, CA (US); Valerie Lindgren, San Diego, CA (US); Pamela Martinez, San Diego, CA (US)

(21) Appl. No.: 17/949,002

(22) Filed: Sep. 20, 2022

Related U.S. Application Data
(63) Continuation-in-part of application No. 16/520,026, filed on Jul. 23, 2019, now abandoned.
(60) Provisional application No. 62/702,752, filed on Jul. 24, 2018.

(51) Int. Cl. G16H 15/00; G06Q 40/08; G06N 5/022

(52) U.S. Cl. CPC G16H 15/00; G06Q 40/08; G06N 5/022

(57) ABSTRACT

A method, non-transitory computer readable medium, and apparatus that identifies one of a plurality of diagnostic mapping tables based on a diagnostic code associated with one of a plurality of data environment formats in an electronic claim. The diagnostic code associated with one of the plurality of data environment formats is correlated to at least one of a plurality of parts and laterality associated with another one of the plurality of data environment formats based on the identified one of the plurality of diagnostic code mapping tables. One of a plurality of assessment ratings is determined based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated.

### CROSS REFERENCE TO RELATED APPLICATIONS

The present application is a continuation-in-part of U.S. patent application Ser. No. 16/520,026, filed Jul. 23, 2019, entitled “METHODS FOR MANAGING ONE OR MORE UNCORRELATED ELEMENTS IN DATA AND DEVICES THEREOF,” and claims the benefit of U.S. Provisional Patent Application Ser. No. 62/702,752, filed Jul. 24, 2018, the disclosures thereof incorporated by reference herein in their entirety.

### DESCRIPTION OF RELATED ART

This technology generally relates to methods, non-transitory computer readable medium, and devices for managing one or more uncorrelated elements in data.

### BACKGROUND

Current estimates predict that the amount of available data is set to reach about 44 zettabytes by 2020. Additionally, in more and more environments, a variety of different types of applications are identifying and providing this available data in response to particular electronic requests and other operations. Often this accessed data has valuable information to assist with the particular electronic requests and other operations, but the accessed data is often uncorrelated and uncategorized for the particular environment. As a result, even though accessible, the provided data is difficult to process and manage and thus does not facilitate the efficient completion of the particular electronic requests and other operations.

By way of example only, in the insurance industry an electronic claim may be received for processing of a claim for physical therapy treatment that includes a diagnosis code: M43.06 Spondylolysis, lumbar region. Spondylolysis is a crack or stress fracture of the vertebrae. This is a condition that most often occurs in children or athletes who participate in sports that involve repeated stress on the back. Unfortunately, in the auto casualty insurance environment, computing devices to assist with the electronic processing of invoices are unable for example to manage how to process this diagnostic code data to determine whether this diagnosis is related to a motor vehicle accident or not in an electronic claim. As a result, prior computing devices in this automated insurance claims processing environment may, for example, require operator input to manually evaluate the diagnostic code to make a determination which is time consuming and expensive or may incorrectly approve or deny the claim as part of the automated processing without proper evaluation of the available diagnostic code data.

Further by way of example only, these prior computing devices in this automated insurance claims processing environment are unable to properly categorize the available data. Currently, all existing severity of injury scales: Abbreviated Injury Scale; Organ Injury Scales; Injury Severity Score; New Injury Severity Score; and International Classification of Diseases (ICD) Injury Severity Score, have an emergency room focus. In other words, these scales were developed to assess morbidity and mortality in an emergency and are not correlated with data in other environments. Unfortunately, there are no severity of injury scales for ICD diagnosis codes that are specific to the auto casualty or workers compensation insurance industry complicating the efficient processing of electronic claims.

### SUMMARY

A method identifies, by a computing apparatus, one of a plurality of diagnostic mapping tables based on a diagnostic code associated with one of a plurality of data environment formats in an electronic claim. The diagnostic code associated with one of the plurality of data environment formats is correlated, by the computing apparatus, to at least one of a plurality of parts and laterality associated with another one of the plurality of data environment formats based on the identified one of the plurality of diagnostic code mapping tables. One of a plurality of assessment ratings is determined, by the computing apparatus, based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated by the computing apparatus.

A non-transitory computer readable medium having stored thereon instructions comprising executable code which when executed by one or more processors, causes the one or more processors to identify one of a plurality of diagnostic mapping tables based on a diagnostic code associated with one of a plurality of data environment formats in an electronic claim. The diagnostic code associated with one of the plurality of data environment formats is correlated to at least one of a plurality of parts and laterality associated with another one of the plurality of data environment formats based on the identified one of the plurality of diagnostic code mapping tables. One of a plurality of assessment ratings is determined based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated.

A computing apparatus includes a memory coupled to a processor which is configured to be capable of executing programmed instructions stored in the memory to identify one of a plurality of diagnostic mapping tables based on a diagnostic code associated with one of a plurality of data environment formats in an electronic claim. The diagnostic code associated with one of the plurality of data environment formats is correlated to at least one of a plurality of parts and laterality associated with another one of the plurality of data environment formats based on the identified one of the plurality of diagnostic code mapping tables. One of a plurality of assessment ratings is determined based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated.

This technology provides a number of advantages including providing methods, non-transitory computer readable medium, and devices for more effective and efficient managing one or more uncorrelated elements in data in different data environment formats for automated electronic claims processing. Additionally, this technology provides a clinically accurate diagnosis code to body part mapping including determination of laterality (side of body) for electronic third party auto bill review software. Further, this technology is able to accurately categorize and assign a severity of injury to a diagnosis code for electronic claims processing. Even further, this technology is able to translate this mapping and categorizations between different types of electronic bill review software which was not previously possible with prior software solutions.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 is a block diagram of an environment with an example of a computing apparatus that maps and categorizes one or more uncorrelated elements in data for automated electronic claims processing;

FIG. 2 is a block diagram of the example of the computing apparatus shown in FIG. 1;

FIG. 3 is a flow chart of an example of a method for managing one or more uncorrelated elements in data for automated electronic claims processing;

FIG. 4 is a table of an example of severity of injury categories for correlation with diagnostic code data;

FIG. 5 is a table of an example of a body part codes and corresponding descriptions; and

FIG. 6 is a table of an example of laterality identification codes and corresponding narratives.

FIG. 7 is an example computing component that may be used to implement various features of embodiments described in the present disclosure.

The figures are not exhaustive and do not limit the present disclosure to the precise form disclosed.

### DETAILED DESCRIPTION

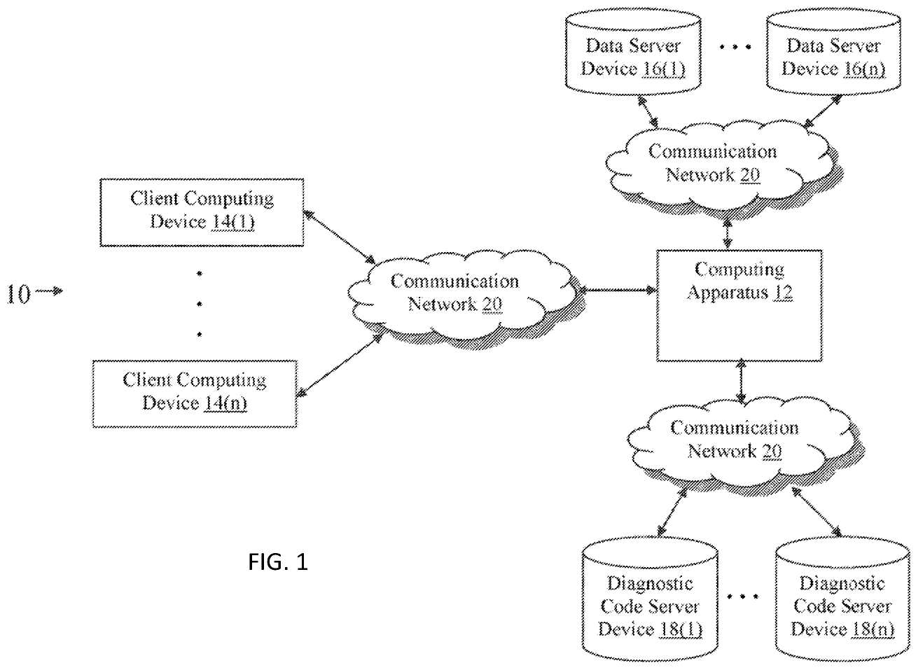

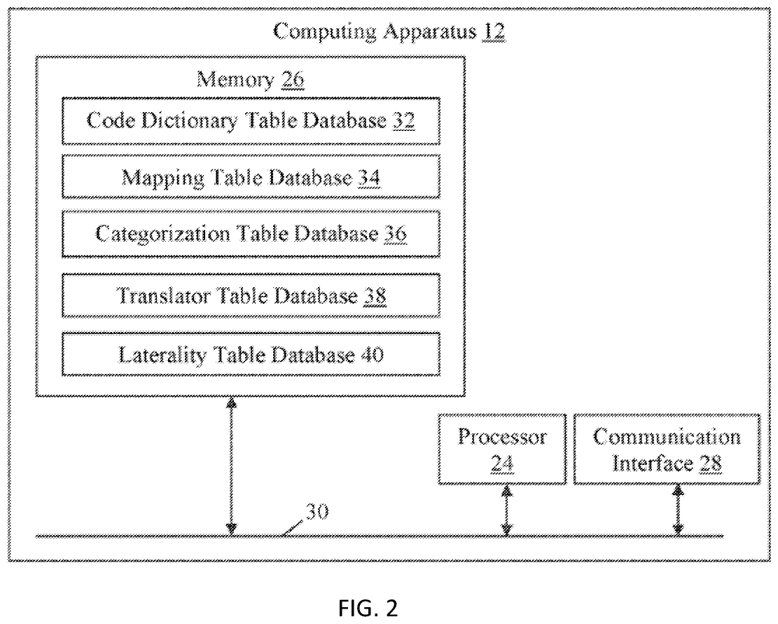

An environment 10 with an example of a computing apparatus 12 that maps and categorizes one or more uncorrelated elements in data for automated electronic claims processing is illustrated in FIGS. 1-2. In this particular example, the environment 10 includes the computing apparatus 12, client computing devices 14(1)-14(n), data server devices 16(1)-16(n), and diagnostic code server devices 18(1)-18(n) coupled via one or more communication networks 20, although the environment could include other types and numbers of systems, devices, components, and/or other elements as is generally known in the art and will not be illustrated or described herein. This technology provides a number of advantages including providing methods, non-transitory computer readable medium, and apparatuses for more effective and efficient managing one or more uncorrelated elements in different data environment formats for automated electronic claims processing.

Referring more specifically to FIGS. 1-2, the computing apparatus 12 is programmed to map and categorize one or more uncorrelated elements in data as illustrated and described herein, although the apparatus can perform other types and/or numbers of functions or other operations and this technology can be utilized with other types of claims. In this particular example, the computing apparatus 12 includes a processor 24, a memory 26, and a communication interface 28 which are coupled together by a bus 30, although the computing apparatus 12 may include other types and/or numbers of physical and/or virtual systems, devices, components, and/or other elements in other configurations.

The processor 24 in the computing apparatus 12 may execute one or more programmed instructions stored in the memory 26 to map and categorize one or more uncorrelated elements in data as illustrated and described in the examples herein, although other types and numbers of functions and/or other operation can be performed. The processor 24 in the computing apparatus 12 may include one or more central processing units and/or general purpose processors with one or more processing cores, for example.

The memory 26 in the computing apparatus 12 stores the programmed instructions and/or other data for one or more aspects of the present technology as described and illustrated herein, although some or all of the programmed instructions and/or data could be stored and/or executed or obtained elsewhere. A variety of different types of memory storage devices, such as a random access memory (RAM) or a read only memory (ROM) in the system or a floppy disk, hard disk, CD ROM, DVD ROM, or other computer readable medium which is read from and written to by a magnetic, optical, or other reading and writing system that is coupled to the processor 24, can be used for the memory 26. In this particular example, the memory 26 includes a code dictionary table database 32, a mapping table database 34, a categorization table database 36, and a translator table database 38, although the memory 26 can comprise other types and/or numbers of other modules, programmed instructions and/or data.

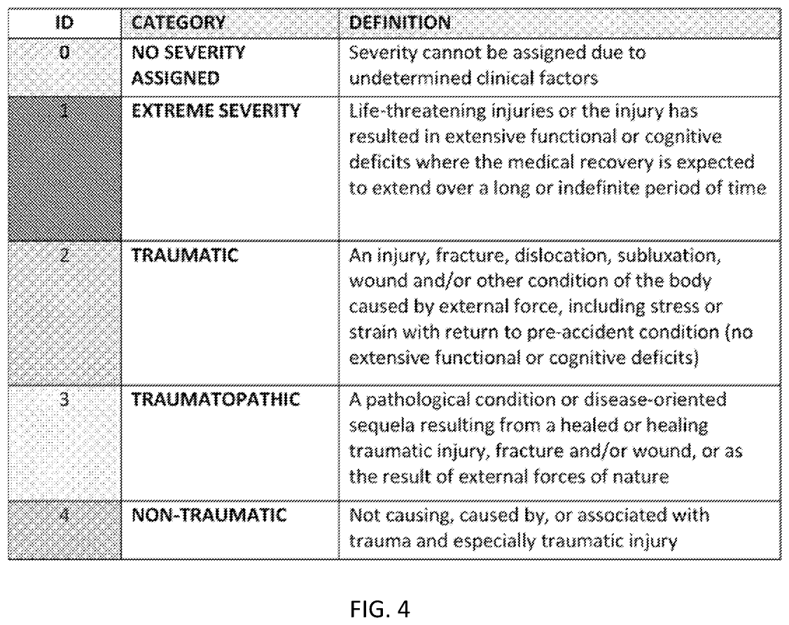

The code dictionary table database 32 may include diagnostic codes, such as ICD-9-CM diagnosis codes and ICD-10-CM diagnosis codes by way of example only, although other types and/or numbers of other codes or other designators in other types of industries or environments with one or more uncorrelated elements in data may be used, such as include ICD-11-CM diagnosis codes by way of example. The mapping table database 34 may include a stored mapping of different formats of diagnosis codes to body part mapping that may also include a laterality (side of body) assignment, although other types and/or number of other correlating mechanisms may be used. The categorization table database 36 may include a correlation of diagnosis codes to a severity of injury for each of the different formats which in this example is specific to and uniquely customized for the auto casualty or workers compensation insurance industry. By way of example, the categorization table database 36 for categorizing ICD diagnosis codes into one of five categories is illustrated in FIG. 4. In this particular example, the categories may comprise: 0—NO SEVERITY ASSIGNED—Severity cannot be assigned due to undetermined clinical factors; 1—EXTREME SEVERITY—Life-threatening injuries or the injury has resulted in extensive functional or cognitive deficits where the medical recovery is expected to extend over a long or indefinite period of time; 2—TRAUMATIC—An injury, fracture, wound and/or other condition of the body caused by external force, including stress or strain with return to pre-accident condition (no extensive functional or cognitive deficits); 3—TRAUMATOPATHIC—A pathological condition or disease-oriented sequela resulting from a healed or healing traumatic injury, fracture and/or wound, or as the result of external forces of nature; and 4—NON-TRAUMATIC—Not causing, caused by, or associated with trauma and especially traumatic injury, although other types and/or numbers of other categorizations or other ratings may be used. The translator table database 38 may, for example, provide a translation from a mapping in a workers compensation format to a mapping in an auto casualty industry format, although other types and/or numbers of cross translation techniques between other formats may be used. The laterality table database 40 may, for example, provide laterality identification, such as not applicable, left side, right side, bilateral and unilateral by way of example only, although other types and/or amounts of laterality identifications may be used Further examples of the programmed instructions and/or data in the code dictionary table database 32, the mapping table database 34, the categorization table database 36, the translator table database 38, and the laterality table database 40 are illustrated and described by way of the examples herein.

The communication interface 28 in the computing apparatus 12 operatively couples and communicates between one or more of the client computing devices 14(1)-14(n), the data server devices 16(1)-16(n), and the diagnostic code server devices 18(1)-18(n), which are all coupled together by one or more of the communication networks 20, although other types and numbers of communication networks or systems with other types and numbers of connections and configurations to other devices and elements. By way of example only, the communication networks 20 can use TCP/IP over Ethernet and industry-standard protocols, including NFS, CIFS, SOAP, XML, LDAP, SCSI, and SNMP, although other types and numbers of communication networks, can be used. The communication networks 20 in this example may employ any suitable interface mechanisms and network communication technologies, including, for example, any local area network, any wide area network (e.g., Internet), teletraffic in any suitable form (e.g., voice, modem, and the like), Public Switched Telephone Network (PSTNs), Ethernet-based Packet Data Networks (PDNs), and any combinations thereof and the like.

Each of the client computing devices 14(1)-14(n) may request unprocessed electronic claims, such as auto casualty electronic claims or workers compensation electronic claims by way of example only, from the computing apparatus 12 which may retrieve from a corresponding one of the data server devices 18(1)-18(n), although the data can be obtained in other manners and/or from other sources. Each of the client computing devices 14(1)-14(n) may request other types of data and/or instructions and may perform other types and/or numbers of other functions and/or operations.

Each of the data server devices 16(1)-16(n) may manage and store unprocessed electronic claims, such as auto casualty electronic claims or workers compensation electronic claims by way of example only, although each of the data server devices may store other types and/or amounts of programmed instructions and/or data. Additionally, each of the diagnostic code server devices 18(1)-18(n) may store and provide requested information and/or other content about diagnostic codes, such as ICD codes by way of example only, although each of the diagnostic code server devices may store other types and/or amounts of programmed instructions and/or data. The computing apparatus 12 may interact with each of the data server devices 16(1)-16(n) and/or each of the diagnostic code server devices 18(1)-18(n) via one or more of the communication networks 20, for example, although other types and/or numbers of storage media in other configurations with other stored information could be used. Each of the data server devices 16(1)-16(n) and/or each of the diagnostic code server devices 18(1)-18(n) also may comprise various combinations and types of storage hardware and/or software and represent a system with multiple network server devices in a data storage pool, which may include internal or external networks. Various network processing applications, such as CIFS applications, NFS applications, HTTP Web Network server device applications, and/or FTP applications, may be operating on each of the data server devices 16(1)-16(n) and/or each of the diagnostic code server devices 18(1)-18(n) and may transmit data in response to requests from the computing apparatus 12.

Each of the client computing devices 14(1)-14(n), each of the data server devices 16(1)-16(n), and each of the diagnostic code server devices 18(1)-18(n) may include a processor, a memory, and a communication interface, which are coupled together by a bus or other link, although other type and/or numbers of other devices and/or nodes as well as other network elements could be used.

Although the exemplary network environment 10 with the computing apparatus 12, the client computing devices 14(1)-14(n), the data server devices 16(1)-16(n), the diagnostic code server devices 18(1)-18(n), and the communication networks 20 are described and illustrated herein, other types and numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

In addition, two or more computing systems or devices can be substituted for any one of the systems or devices in any example. Accordingly, principles and advantages of distributed processing, such as redundancy and replication also can be implemented, as desired, to increase the robustness and performance of the devices, apparatuses, and systems of the examples. The examples may also be implemented on computer system(s) that extend across any suitable network using any suitable interface mechanisms and traffic technologies, including by way of example only teletraffic in any suitable form (e.g., voice and modem), wireless traffic media, wireless traffic networks, cellular traffic networks, G3 traffic networks, Public Switched Telephone Network (PSTNs), Packet Data Networks (PDNs), the Internet, intranets, and combinations thereof.

The examples also may be embodied as a non-transitory computer readable medium having instructions stored thereon for one or more aspects of the present technology as described and illustrated by way of the examples herein, as described herein, which when executed by the processor, cause the processor to carry out the steps necessary to implement the methods of this technology as described and illustrated with the examples herein.

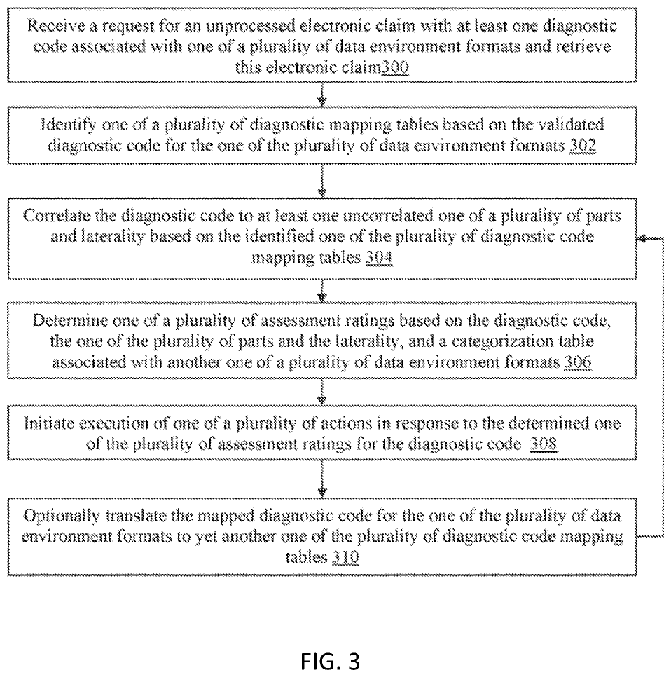

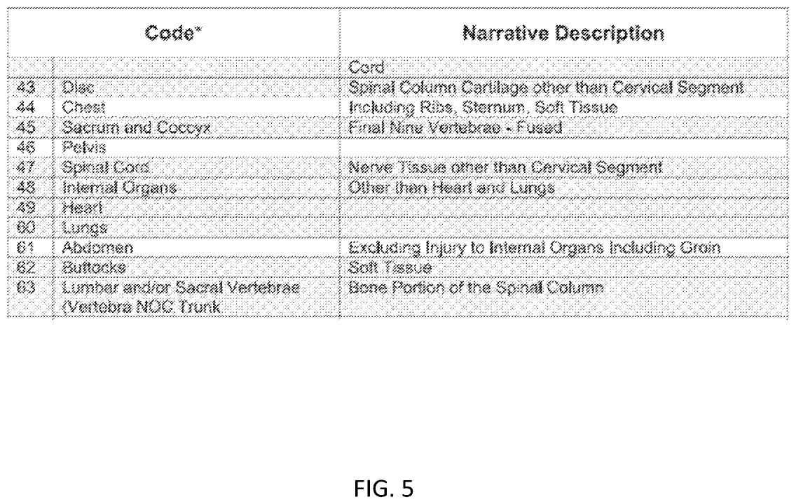

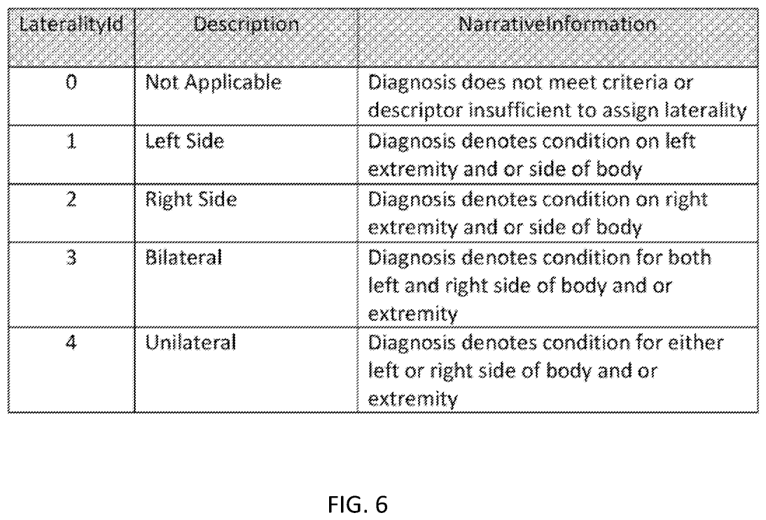

An example of a method for managing one or more uncorrelated elements in data will now be described with reference to FIGS. 1-6. Referring more specifically to FIG. 3, in step 300 the computing apparatus 12 may receive a request for an unprocessed electronic claim from one of the plurality of client computing devices 14(1)-14(n), such as a request for an unprocessed electronic workers compensation claim or an electronic third party auto casualty claim by way of example only. The electronic claim may have at least one diagnostic code associated with one of a plurality of data environment formats. Based on the received request, the computing apparatus 12 may retrieve this unprocessed electronic claim from one of the data server devices 16(1)-16(n) which may store electronic worker compensation claims or electronic auto casualty claims for processing by one of the client computing devices 14(1)-14(n) by way of example only, although other types and/or amounts of data may be stored. By way of a further example only, the electronic claim may be an electronic workers compensation claim for physical therapy treatment that includes a diagnosis code: M43.06 Spondylolysis, lumbar region.

The computing apparatus 12 may also determine if the at least one diagnostic code in the electronic claim is one of a plurality of valid diagnostic codes for the one of the plurality of data environment formats. In this example, the computing apparatus 12 may determine if the one of the ICD is one of a plurality of valid ICD diagnostic codes stored in this example in the code dictionary table database 32 for the one of the plurality of data environment formats, although the diagnostic code can be validated in other manners. If the at least one diagnostic code in the electronic claim is determined by the computing apparatus 12 to be invalid, then an electronic communication rejecting the electronic claim may be transmitted, although other types of actions may be taken. This external automated validation can streamline and prevent one of the client computing devices 14(1)-14(n) from working on an invalid electronic claim.

In step 302, the computing apparatus 12 may identify one of a plurality of diagnostic mapping tables stored in mapping table database 34, in this example, based on the validated diagnostic code for the one of the plurality of data environment formats. Each of the diagnostic mapping tables has one or more associated coding formats for another one of the plurality of data environment formats that can be correlated to the validated diagnostic code by the computing apparatus 12.

In step 304, the computing apparatus 12 may map the received diagnostic code for the one of the plurality of data environment formats in the retrieved claim data to a numeric or other identifier for at least one of a plurality of parts, such as human body part data by way of example only, for another one of the plurality of data environment formats based on the identified one of the plurality of diagnostic code mapping tables. By way of example, the computing apparatus 12 may map the received diagnostic code M43.06 to the NcciBodyPartId numeric identifier ‘63’ which refers to a body part: the Vertebrae—Thoracic/Lumbar/Sacral region of the body using the example of a portion of a mapping table stored in the mapping table database 34 as shown in Table 1.

TABLE 1

| Diagnosis Code | lcd Version | Start Date | NcciBodyPartId |
|---|---|---|---|
| M43.06 | 10 | 2015 Oct. 1 | 63 |
| | | 00; 0 . . . | |

In step 304, the computing apparatus 12 may also use the mapping table to determine a particular location or laterality, i.e. side of the body, of the identified body part or other region, which laterality data also may be from a different one of the plurality of data environment formats, although other types and/or other numbers of uncorrelated data elements in the other ones of the plurality of data environment formats may be correlated to the diagnostic code in the one of the plurality of data environment formats. In this example, each of the diagnostic codes may have a laterality indication stored in the stored mapping table from the database 34, although other manners for determining laterality may be used. By way of further example, the mapping table may provide a corresponding LateralityId numeric identifier ‘0’ which for this example indicates that laterality for diagnosis code M43.06 does not apply (i.e. diagnostic code does not meet criteria or is insufficient to indicate laterality). An illustration of a portion of a mapping table stored in the mapping table database 34 which illustrates this is provided in Table 2.

TABLE 2

| Diagnosis | | | | | | | | | | Laterality |
|---|---|---|---|---|---|---|---|---|---|---|
| Code | Ic . . . | Star . . . | En . . . | No . . . | Tra . . . | D . . . | Description | Di . . . | Di . . . | Id |
| M43.06 | 10 | 2015 . . . | 2999 . . . | False | False | 0 | Spondylolysis, | Nu . . . | 4 | 0 |
| | | | | | | | lumbar | | | |
| | | | | | | | region | | | |

In other examples, the laterality may be determined in other manners, such as with laterality information stored in laterality table database 40 that can be correlated to the identified body part and/or based on other information in the claim being processed by the computing apparatus.

In step 306, the computing apparatus 12 may determine one of a plurality of assessment ratings based on the validated diagnostic code, the one of the plurality of parts, the laterality and a categorization table for the another one of the plurality of data environment formats as illustrated in FIG. 4, although other types and/or numbers of factors may be to determine the assessment and/or other types of categorizations may be used. In the example above for the electronic workers compensation claim for physical therapy treatment, the computing apparatus 12 may determine that for the validated diagnosis code: M43.06 Spondylolysis, the part is the lumbar region and the laterality is none and based on the categorization table and information in the electronic claim associated with the diagnostic code that the assessment is a DiagnosisSeverityId rating assessment of 4-Non-traumatic (Not causing, caused by, or associated with trauma and especially traumatic injury).

Referring again to FIG. 3, the process 300 may include providing identifiers of the single buckets as input to a machine learning model, at 306. In the example of FIG. 2, the bill triage tool 216 may provide the identifiers to one or more of the machine learning models 218.

In some embodiments, determining the assessment ratings may include the use of one or more trained machine learning models. Any machine learning models may be used. For example, the machine learning models and techniques may include classifiers, decision trees, neural networks, gradient boosting, and similar machine learning models and techniques.

In some embodiments, the disclosed technologies may include the use of one or more trained machine learning models at one or more points in the described processes. Any machine learning models may be used. For example, the machine learning models and techniques may include classifiers, decision trees, neural networks, gradient boosting, and similar machine learning models and techniques. Different iterations may employ the same trained machine learning model and/or different trained machine learning models. For example, a first iteration may employ a cosine similarity or machine model. A second iteration may employ an auto encoder, STOSA, or machine model. A third iteration may employ a group NN or machine model. Subsequent iterations may employ a STOSA or machine model.

The machine learning models may be trained previously according to historical correspondences between input parameters and corresponding assessments. The input parameters may include those described above, for example such as validated diagnostic code, the one of the plurality of parts, the laterality, and the categorization table. Once the machine learning models have been trained, new input parameters may be applied to the trained machine learning model as inputs. In response, the machine learning models may provide the assessments as outputs.

Some embodiments include the training of the machine learning models. The training may be supervised, unsupervised, or a combination thereof, and may continue between operations for the lifetime of the system. The training may include creating a training set that includes the input parameters and corresponding assessments described above.

The training may include one or more second stages. A second stage may follow the training and use of the trained machine learning models, and may include creating a second training set, and training the trained machine learning models using the second training set. The second training set may include the inputs applied to the machine learning models, and the corresponding outputs generated by the machine learning models, during actual use of the machine learning models.

The second training stage may include identifying erroneous assessments generated by the machine learning model, and adding the identified erroneous assessments to the second training set. Creating the second training set may also include adding the inputs corresponding to the identified erroneous assessments to the second training set.

By way of example, the computing apparatus 12 may optionally use a machine learning model that may utilize deep learning to store previously analyzed data related to corresponding diagnostic codes and may develop and refine an algorithm or other executable rule or rules to further assist with determining the assessment rating based on one or more of the factors discussed in the example above. An example of the results of this assessment are illustrated in a portion of Table 3.

TABLE 3

| | | | | | | | | | Diagnosis | |
|---|---|---|---|---|---|---|---|---|---|---|
| Diagnosis | | | | | | | | | Severity | Laterality |
| Code | Ic . . . | Star . . . | En . . . | No . . . | Tra . . . | D . . . | Description | Di . . . | Id | Id |
| M43.06 | 10 | 2015 . . . | 2999 . . . | False | False | 0 | Spondylolysis, | Nu . . . | 4 | 0 |
| | | | | | | | lumbar | | | |
| | | | | | | | region | | | |

With this determined assessment rating, the electronic claim can more quickly and accurately be processed by the computing apparatus 12 saving valuable time, eliminating the expense of an external review and an inaccurate payment or other disposition of the electronic claim.

In step 308, the computing apparatus 12 may initiate execution of one of a plurality of actions in response to the determined one of the plurality of assessment ratings for the diagnostic code. By way of example only, the computing apparatus 12 may initiate an action to transmit an acceptance or rejection of the electronic claim or to initiate an action to electronically request additional data or a further evaluation, although other types and/or numbers of actions may be initiated.

In the example being used herein, for the diagnosis code M43.06 where DiagnosisSeverityId is indicated as ‘4’ (non-traumatic), the computing apparatus 12 may deny reimbursement of the electronic claim as this diagnostic code has no possibility of accident relatedness. Accordingly, with this automated management of one or more uncorrelated elements in data, the claimed technology provides a much higher accuracy and consistency at a faster rate when electronic processing of claims. Based on the determined ratings assessment, if a diagnosis code has been assigned a value=3 (TRAUMATOPATHIC: A pathological condition or disease-oriented sequela resulting from a healed or healing traumatic injury, fracture and/or wound, or as the result of external forces of nature), an automated alert may be initiated to alert the user that review of medical records and/or billing may be warranted to confirm accident relatedness.

In step 310, the computing apparatus 12 may optionally translate the mapped diagnostic code for the one of the plurality of data environment formats to yet another one of the plurality of diagnostic code mapping tables, such as a translation from a workers compensation mapping to a third party auto casualty mapping, although other types of translations may be completed. A portion of an example diagnostic code mapping table is shown in Table 4. In this example, for the diagnosis code M43.06, NcciBodyPartId ‘63’ which refers to the Vertebrae—Thoracic/Lumbar/Sacral region of the body, once the NcciBodyPartId is identified as described earlier, the computing apparatus 12 may translate the NcciBodyPartId to a HybridBodyPartId that is specific to the auto casualty market via a translator table in the translator table database utilizing the determination and assessment from the prior evaluation. A portion of an example translator table is shown in Table 5. In this example, the HybridBodyPartId ‘4001’ referenced is the Upper Back (Thoracic) or ‘4003’ Lower Back (Lumbar—Coccyx) region of the body.

TABLE 4

| Diagnosis Code | lcdVersion | StartDate | NcciBodyPartid |
|---|---|---|---|
| M43.06 | 10 | 2015 Oct. 1 | 63 |
| | | 00: 0 . . . | |

TABLE 5

| HybridBodyPa . . . | Description |
|---|---|
| 4001 | Upper Back (Thoracic) |
| 4003 | Lower Back (Lumbar - Coccyx) |

Accordingly, in this example the computing apparatus 12 may use the stored translation data table to translate this value (NcciBodyPartId=63) to an auto-casualty specific diagnosis body part mapping (HybridBodyPartId=4003) and then may for example optional proceed back to step 304 as described above to automatically process the diagnostic code in the electronic claim in the same manner as described in the example above, although the claim may be processed in other manners.

Thus, as illustrated and described by way of the examples herein, this technology provides more effective and efficient management of one or more uncorrelated elements in data in different data environment formats for automated electronic claims processing. Additionally, this technology provides an automated clinically accurate diagnosis code to body part mapping including determination of laterality (side of body) for electronic third party auto bill review software. Further, this technology is able to accurately categorize and assign a severity of injury to a diagnosis code for electronic claims processing. Even further, this technology is able to automatically translate this management of uncorrelated data between different types of electronic claim formats.

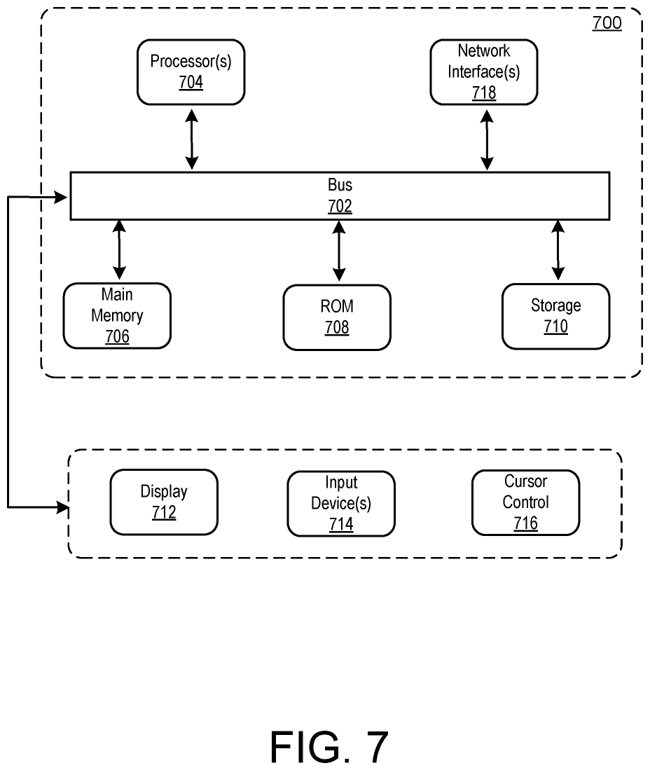

FIG. 7 depicts a block diagram of an example computer system 700 in which embodiments described herein may be implemented. The computer system 700 includes a bus 702 or other communication mechanism for communicating information, one or more hardware processors 704 coupled with bus 702 for processing information. Hardware processor(s) 704 may be, for example, one or more general purpose microprocessors.

The computer system 700 also includes a main memory 706, such as a random access memory (RAM), cache and/or other dynamic storage devices, coupled to bus 702 for storing information and instructions to be executed by processor 704. Main memory 706 also may be used for storing temporary variables or other intermediate information during execution of instructions to be executed by processor 704. Such instructions, when stored in storage media accessible to processor 704, render computer system 700 into a special-purpose machine that is customized to perform the operations specified in the instructions.

The computer system 700 further includes a read only memory (ROM) 708 or other static storage device coupled to bus 702 for storing static information and instructions for processor 704. A storage device 710, such as a magnetic disk, optical disk, or USB thumb drive (Flash drive), etc., is provided and coupled to bus 702 for storing information and instructions.

The computer system 700 may be coupled via bus 702 to a display 712, such as a liquid crystal display (LCD) (or touch screen), for displaying information to a computer user. An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. In some embodiments, the same direction information and command selections as cursor control may be implemented via receiving touches on a touch screen without a cursor.

The computing system 700 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “component,” “engine,” “system,” “database,” data store,” and the like, as used herein, can refer to logic embodied in hardware or firmware, or to a collection of software instructions, possibly having entry and exit points, written in a programming language, such as, for example, Java, C or C++. A software component may be compiled and linked into an executable program, installed in a dynamic link library, or may be written in an interpreted programming language such as, for example, BASIC, Perl, or Python. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

The computer system 700 may implement the techniques described herein using customized hard-wired logic, one or more ASICs or FPGAs, firmware and/or program logic which in combination with the computer system causes or programs computer system 700 to be a special-purpose machine. According to one embodiment, the techniques herein are performed by computer system 700 in response to processor(s) 704 executing one or more sequences of one or more instructions contained in main memory 706. Such instructions may be read into main memory 706 from another storage medium, such as storage device 710. Execution of the sequences of instructions contained in main memory 706 causes processor(s) 704 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 710. Volatile media includes dynamic memory, such as main memory 706. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 702. Transmission media can also take the form of acoustic or light waves, such as those generated during radio-wave and infra-red data communications.

The computer system 700 also includes a communication interface 718 coupled to bus 702. Network interface 718 provides a two-way data communication coupling to one or more network links that are connected to one or more local networks. For example, communication interface 718 may be an integrated services digital network (ISDN) card, cable modem, satellite modem, or a modem to provide a data communication connection to a corresponding type of telephone line. As another example, network interface 718 may be a local area network (LAN) card to provide a data communication connection to a compatible LAN (or a WAN component to communicate with a WAN). Wireless links may also be implemented. In any such implementation, network interface 718 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet.” Local network and Internet both use electrical, electromagnetic or optical signals that carry digital data streams. The signals through the various networks and the signals on network link and through communication interface 718, which carry the digital data to and from computer system 700, are example forms of transmission media.

The computer system 700 can send messages and receive data, including program code, through the network(s), network link and communication interface 718. In the Internet example, a server might transmit a requested code for an application program through the Internet, the ISP, the local network and the communication interface 718.

The received code may be executed by processor 704 as it is received, and/or stored in storage device 710, or other non-volatile storage for later execution.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computers processors, not only residing within a single machine, but deployed across a number of machines.

As used herein, a circuit might be implemented utilizing any form of hardware, or a combination of hardware and software. For example, one or more processors, controllers, ASICs, PLAs, PALs, CPLDs, FPGAs, logical components, software routines or other mechanisms might be implemented to make up a circuit. In implementation, the various circuits described herein might be implemented as discrete circuits or the functions and features described can be shared in part or in total among one or more circuits. Even though various features or elements of functionality may be individually described or claimed as separate circuits, these features and functionality can be shared among one or more common circuits, and such description shall not require or imply that separate circuits are required to implement such features or functionality. Where a circuit is implemented in whole or in part using software, such software can be implemented to operate with a computing or processing system capable of carrying out the functionality described with respect thereto, such as computer system 700.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

The foregoing description of the present disclosure has been provided for the purposes of illustration and description. It is not intended to be exhaustive or to limit the disclosure to the precise forms disclosed. The breadth and scope of the present disclosure should not be limited by any of the above-described exemplary embodiments. Many modifications and variations will be apparent to the practitioner skilled in the art. The modifications and variations include any relevant combination of the disclosed features. The embodiments were chosen and described in order to best explain the principles of the disclosure and its practical application, thereby enabling others skilled in the art to understand the disclosure for various embodiments and with various modifications that are suited to the particular use contemplated. It is intended that the scope of the disclosure be defined by the following claims and their equivalence.

What is claimed is:

1. A method comprising:
receiving, by a computing apparatus from a client device, a request to process an electronic claim comprising a diagnostic code associated with a treatment procedure in one of a plurality of data environment formats;
retrieving, by the computing apparatus, the electronic claim specified by the request;
identifying, by the computing apparatus, one of a plurality of diagnostic mapping tables by correlating the diagnostic code in the one of the plurality of data environment formats in the electronic claim to one or more coding formats associated with the one of the plurality of diagnostic mapping tables;
determining, by the computing apparatus, first and second identifiers corresponding to the diagnostic code in the one of the plurality of data environment formats based on the identified one of diagnostic mapping tables, wherein the first identifier represents at least one of a plurality of human body parts and the second identifier represents laterality of the at least one human body part;
determining, by the computing apparatus, one of a plurality of assessment ratings based on input parameters, wherein the input parameters comprise at least one of the diagnostic code, the determined one of the plurality of the human body parts specified by the first identifier, the determined laterality specified by the second identifier, and a categorization table associated with another one of the plurality of data environment formats, wherein determining the one of a plurality of assessment ratings based on input parameters comprises applying the input parameters as inputs to the trained machine learning model, wherein responsive to the inputs, the trained machine learning model outputs the one of a plurality of assessment ratings, and wherein the trained machine learning model has been trained using historical correspondences between the input parameters and corresponding assessments; and
initiating, by the computing apparatus, execution of one of a plurality of actions on the electronic claim in response to the determined one of the plurality of assessment ratings for the diagnostic code.

2. The method of claim 1, further comprising:
creating a first training set comprising the historical correspondences between the input parameters and corresponding assessments; and
training the machine learning model using the first training set.

3. The method of claim 2, further comprising:
creating a second training set comprising the correspondences between the input parameters applied to the trained machine learning model and corresponding assessments produced by the machine learning model; and
training the machine learning model using the second training set.

4. The method of claim 3, further comprising:
identifying erroneous assessments generated by the machine learning model;
adding the identified erroneous assessments to the second training set; and
training the machine learning model using the second training set after adding the identified erroneous assessments to the second training set.

5. The method of claim 1, wherein the determining the one of the plurality of assessment ratings is further based on data in the electronic claim related to the diagnostic code.

6. The method of claim 1, further comprising:
determining, by the computing apparatus, whether the diagnostic code in the received request is one of a plurality of valid diagnostic codes for the one of the plurality of data environment formats;
wherein the initiating execution of one of a plurality of actions on the electronic claim further comprises transmitting an electronic communication rejecting the electronic claim when the diagnostic code is determined not to be one of the plurality of valid diagnostic codes for the one of the plurality of data environment formats.

7. The method of claim 1, further comprising translating, by the computing apparatus, the diagnostic code to another one of the plurality of diagnostic mapping tables associated with yet another one of the plurality of data environment formats.

8. A non-transitory computer readable medium having stored thereon instructions comprising executable code which when executed by one or more processors, causes the one or more processors to:
receive, from a client device, a request to process an electronic claim comprising a diagnostic code associated with a treatment procedure in one of a plurality of data environment formats;
retrieve the electronic claim specified by the request;
identify one of a plurality of diagnostic mapping tables by correlating the diagnostic code in one of the plurality of data environment formats in the electronic claim to one or more coding formats associated with the one of the plurality of diagnostic mapping tables;
determine first and second identifiers corresponding to the diagnostic code in the one of the plurality of data environment formats based on the identified one of diagnostic mapping tables, wherein the first identifier represents at least one of a plurality of human body parts and the second identifier represents laterality of the one human body part;
determine, by the computing apparatus, one of a plurality of assessment ratings based on input parameters, wherein the input parameters comprise at least one of the diagnostic code, the determined one of the plurality of the human body parts specified by the first identifier, the determined laterality specified by the second identifier, and a categorization table associated with another one of the plurality of data environment formats, wherein determining the one of a plurality of assessment ratings based on input parameters comprises applying the input parameters as inputs to the trained machine learning model, wherein responsive to the inputs, the trained machine learning model outputs the one of a plurality of assessment ratings, and wherein the trained machine learning model has been trained using historical correspondences between the input parameters and corresponding assessments; and
initiate execution of one of a plurality of actions on the electronic claim in response to the determined one of the plurality of assessment ratings for the diagnostic code.

9. The non-transitory computer readable medium of claim 8, wherein the executable code when executed by the one or more processors further causes the one or more processors to:
create a first training set comprising the historical correspondences between the input parameters and corresponding assessments; and
train the machine learning model using the first training set.

10. The non-transitory computer readable medium of claim 8, wherein the executable code when executed by the one or more processors further causes the one or more processors to:
create a second training set comprising the correspondences between the input parameters applied to the trained machine learning model and corresponding assessments produced by the machine learning model; and
train the machine learning model using the second training set.

11. The non-transitory computer readable medium of claim 8, wherein the executable code when executed by the one or more processors further causes the one or more processors to:
identify erroneous assessments generated by the machine learning model;
add the identified erroneous assessments to the second training set; and
train the machine learning model using the second training set after adding the identified erroneous assessments to the second training set.

12. The non-transitory computer readable medium of claim 8, wherein the determine the one of the plurality of assessment ratings is further based on data in the electronic claim related to the diagnostic code.

13. The non-transitory computer readable medium of claim 8, wherein the assessment ratings include a classification of diagnostic codes wherein the executable code when executed by the one or more processors further causes the one or more processors to:
determine when the diagnostic code in the received request is one of a plurality of valid diagnostic codes for the one of the plurality of data environment formats;
wherein the initiating execution of one of a plurality of actions on the electronic claim further comprises transmitting an electronic communication rejecting the electronic claim when the diagnostic code is determined not to be one of the plurality of valid diagnostic codes for the one of the plurality of data environment formats.

14. The non-transitory computer readable medium of claim 8, wherein the executable code when executed by the one or more processors further causes the one or more processors to:
translate the diagnostic code to another one of the plurality of diagnostic mapping tables associated with yet another one of the plurality of data environment formats.

15. A computing apparatus comprising:
a processor; and
a memory coupled to the processor which is configured to be capable of executing programmed instructions stored in the memory to:
receive, from a client device, a request to process an electronic claim comprising a diagnostic code associated with a treatment procedure in one of a plurality of data environment formats;
retrieve the electronic claim specified by the request;
identify one of a plurality of diagnostic mapping tables by correlating the diagnostic code in one of the plurality of data environment formats in the electronic claim to one or more coding formats associated with the one of the plurality of diagnostic mapping tables;
determine first and second identifiers corresponding to the diagnostic code in the one of the plurality of data environment formats based on the identified one of diagnostic mapping tables, wherein the first identifier represents at least one of a plurality of human body parts and the second identifier represents laterality of the one human body part;
determine, by the computing apparatus, one of a plurality of assessment ratings based on input parameters, wherein the input parameters comprise at least one of the diagnostic code, the determined one of the plurality of the human body parts specified by the first identifier, the determined laterality specified by the second identifier, and a categorization table associated with another one of the plurality of data environment formats, wherein determining the one of a plurality of assessment ratings based on input parameters comprises applying the input parameters as inputs to the trained machine learning model, wherein responsive to the inputs, the trained machine learning model outputs the one of a plurality of assessment ratings, and wherein the trained machine learning model has been trained using historical correspondences between the input parameters and corresponding assessments; and
initiate execution of one of a plurality of actions on the electronic claim in response to the determined one of the plurality of assessment ratings for the diagnostic code.

16. The computing apparatus of claim 15, wherein the executable code when executed by the one or more processors further causes the one or more processors to:
create a first training set comprising the historical correspondences between the input parameters and corresponding assessments; and
train the machine learning model using the first training set.

17. The computing apparatus of claim 15, wherein the executable code when executed by the one or more processors further causes the one or more processors to:
create a second training set comprising the correspondences between the input parameters applied to the trained machine learning model and corresponding assessments produced by the machine learning model; and
train the machine learning model using the second training set.

18. The computing apparatus of claim 15, wherein the executable code when executed by the one or more processors further causes the one or more processors to:
identify erroneous assessments generated by the machine learning model;
add the identified erroneous assessments to the second training set; and
train the machine learning model using the second training set after adding the identified erroneous assessments to the second training set.

19. The computing apparatus of claim 15, wherein the determine the one of the plurality of assessment ratings is further based on data in the electronic claim related to the diagnostic code.

20. The computing apparatus of claim 15, wherein the executable code when executed by the one or more processors further causes the one or more processors to:
determine when the diagnostic code in the received request is one of a plurality of valid diagnostic codes for the one of the plurality of data environment formats;
wherein the initiating execution of one of a plurality of actions on the electronic claim further comprises transmitting an electronic communication rejecting the electronic claim when the diagnostic code is determined not to be one of the plurality of valid diagnostic codes for the one of the plurality of data environment formats.

21. The computing apparatus of claim 15, wherein the processor coupled to the memory is further configured to be capable of executing at least one additional programmed instruction stored in the memory to:
translate the diagnostic code to another one of the plurality of diagnostic mapping tables associated with yet another one of the plurality of data environment formats.
